(19) United States
(12) Patent Application Publication
(10) Pub. No.: US 2023/0192865 A1
(43) Pub. Date: Jun. 22, 2023

(54) NANO ANTIBODY AND USE THEREOF

(71) Applicant: CROPEDIT BIOTECHNOLOGY INC., Beijing (CN)

(72) Inventors: Zhe SONG, Beijing (CN); Youlin QI, Beijing (CN); Yaobin QIN, Beijing (CN); Jianfeng YANG, Beijing (CN); Limin HOU, Beijing (CN); Zhiwei ZHU, Beijing (CN); Qiuping DONG, Beijing (CN); Xianggan LI, Beijing (CN); Lei ZHANG, Beijing (CN); Jinyu WANG, Beijing (CN); Yuejin LI, Beijing (CN)

(21) Appl. No.: 17/923,797

(22) PCT Filed: May 6, 2020
(86) PCT No.: PCT/CN2020/088668

§ 371 Date: Nov. 7, 2022

(51) Int. Cl. C07K 16/28; A61P 37/06

(52) U.S. Cl. CPC C07K 16/2851; A61P 37/06; C07K 2317/565; C07K 2317/569

(57) ABSTRACT

Provided is a nano antibody, the amino acid sequence thereof being EVQLQASGGGFVQPGGSLRLSCAASGFTFSSX₁AMGWFRQAPGKEREX₂VSAISSGGGNTYYADSVKGRFTISRDNSKNTVYLQMNSLRAEDTATYYCVTPGGRLWYYRYDYRCQGTQVTVSS (SEQ ID NO: 1), where X₁ is selected from Y or F, and X₂ is selected from F or L. The antibody can be used to dissolve Charcot-Leyden crystals (CLCs), thereby reducing pulmonary inflammation, changes in lung function, and mucus production. Further provided is the use of the nano antibody in the preparation of a drug and a reagent for testing Charcot-Leyden crystals (CLCs) and/or Galectin-10 protein.

### CROSS REFERENCE TO RELATED APPLICATIONS AND PRIORITY

This patent application claims priority from PCT Application No. PCT/CN2020/088668 filed May 6, 2020.This application is herein incorporated by reference in its entirety.

### TECHNICAL FIELD

The present invention belongs to the field of biotechnology, and in particular relates to a nano antibody polypeptide sequence that can be used to dissolve eosinophilic airway inflammation marker Galectin-10 crystals (CLCs) in vitro and use thereof.

### BACKGROUND

Asthma, as a chronic inflammatory disease of the airway, manifests clinically as recurrent wheezing, shortness of breath, chest distress or coughing and other symptoms. It often attacks or worsens at night and in the early morning, and most patients can relieve themselves or relieve after treatment, but it seriously affects their quality of life. The development of severe asthma is difficult to control, with frequent acute attacks, resulting in high medical costs, and is also the main cause of disability and death from asthma. In recent years, with the increasing prevalence of asthma worldwide, the research and development of drugs for the effective relief and treatment of asthma has become one of the important goals in the field of pharmaceutical research and development.

At present, the prevention and treatment drugs for asthma mainly involve two directions, that is, the anti-inflammatory effect on airway inflammation and the use of new targets for asthma treatment. Anti-inflammation is the basic treatment method for asthma, but it is not effective for severe patients. Therefore, it is necessary to seek new therapeutic targets to improve asthma symptoms. The existing therapeutic drugs can be divided into non-biological drugs and biological drugs.

The non-biological drugs can be divided into control drugs and relief drugs according to the need for long-term use to control symptoms or as acute relief drugs during acute attacks. The control drugs include inhaled glucocorticoids, leukotriene modifiers, sodium cromoglycate, systemic hormones, long-acting p2-receptor agonists, sustained-release theophylline, etc. that achieve clinical control through anti-inflammatory effects. The relief drugs achieve acute relief of symptoms by rapidly relieving bronchospasm. Such drugs include systemic hormones, inhaled β2-agonists, short-acting β2-agonists, short-acting theophylline, inhaled anticholinergic drugs, etc. The inhaled glucocorticoids benefit most asthma patients, but for some severe, hormone-dependent or resistant patients, their clinical control effect is not good, and requires modulation with adjuvant biological drugs.

The development of biological drugs is based on the understanding of asthma pathophysiology and the target analysis of inflammatory mediators at the cellular and molecular level. The main pathological changes of asthma are completed by respiratory epithelial cells, and inflammatory cells and inflammatory mediators including dendritic cells, B lymphocytes, T lymphocytes, eosinophils, mast cells, basophils, etc. After the body is exposed to external stimuli (such as allergens), the stimuli entering the airway will be taken up and processed by antigen-presenting cells such as dendritic cells and then presented to T lymphocytes, thus prompting T cells to differentiate into helper T cells, namely Th1 and Th2. Th1 secretes cytokines, mainly interleukin 2 (IL-2) and interferon γ (IFN-γ), which can activate cellular immune responses, including the recruitment and activation of natural killer cells, mononuclear macrophages, neutrophils, etc. Under the action of basophils, mast cells and type II innate lymphoid cells (ILC2s) derived from helper cells, T helper cells differentiate into Th2 subsets. Th2 secretes cytokines such as interleukin 4 (IL-4), interleukin 5 (IL-5) and interleukin 13 (IL1-3) to activate the humoral immune response, promote the production of immunoglobulin E (IgE), and enhance the inflammatory response of eosinophils. The existing commercial biological drugs for asthma include: Qmalizumab, Mepolizumab, Reslizumab, Benralizumab and Dupilumab, which involve therapeutic targets IgE, IL-4 and IL-5.

Resolving mucus accumulation in the respiratory tract is one of the potential ways to relieve and treat asthma and rhinitis. Charcot in 1853 and Leyden in 1872 respectively reported the observation of extracellular bipyramidal crystals in the airways of asthmatic patients, which were later named Charcot-Leyden crystals (CLCs). Many researchers have since discovered such crystals in chronic allergic and inflammatory diseases. The crystal is composed of protein Galectin-10 (Gal10).

Galectin-10 is one of the most abundant proteins in eosinophils, which help generate inflammatory responses in the body. Galectin-10 remains largely dissolved in eosinophils, and once it is released as part of immune defenses, it can only form crystals.

As in gout, uric acid crystals cause very painful arthritis, could CLCs stimulate the immune response system in the lungs to create excessive inflammatory responses that lead to diseases? To address this issue, Emma K. Persson et al. in Belgian prepared recombinant Galectin-10 crystals in Escherichia coli, which are structurally and biochemically similar to CLCs obtained from patients with sinusitis and asthma. Meanwhile, soluble and crystal defective Galectin-10 mutant proteins were designed according to the structural characteristics. The immune response in a mouse model of asthma was studied using Galectin-10 in a crystal state and a liquid state respectively, and the results showed that only when Galectin-10 was in the crystal state, it can induce a complete immune response; and in the solution, Galectin-10 was harmless. Most importantly, the Galectin-10 crystals in the form of Charcot-Leyden crystals are the key feature in the induction of asthma, including the production of altered mucus. Subsequently, the research team and a biotechnology company Argenx in Ghent developed an antibody that can specifically resist CLCs. The antibody can dissolve CLCs in a few minutes in a petri dish in a laboratory and within hours in a patient’s ex vivo mucus. In the mouse model of asthma, the antibody can significantly reduce pulmonary inflammation, changes in lung function, and mucus production. The following conclusions were obtained by studies: 1. Galectin-10 is a very abundant protein in eosinophils and basophils, and its formation is closely related to the release of extracellular traps in eosinophils; 2. Galectin-10 crystals stimulate the immune system, produce airway inflammatory responses, and promote the production of main symptoms of asthma such as sputum in the respiratory tract, while the non-crystalline Galectin-10 protein is completely harmless; and 3. The Galectin-10 antibody can quickly dissolve Galectin-10 crystals, and also can suppress airway inflammation, goblet cell metaplasia, bronchial hyperresponsiveness (BHR) and IgE synthesis induced by Galectin-10 crystals in humanized mouse models of asthma.

Therefore, Galectin-10 is a potential target for suppressing asthma, but there are few related technologies and reports on Galectin-10 antibodies that can rapidly dissolve CLCs in vitro.

### SUMMARY

The present invention provides a Galectin-10 antibody, which can rapidly dissolve CLCs in vitro. The antibody disclosed in the present invention is a nano antibody, which has a molecular weight of about ⅒ of that of a common antibody, and also has the advantages of strong tissue penetration and good protein stability, can rapidly dissolve CLCs in vitro, and is expected to become an antibody drug for treating asthma or other diseases related to eosinophilia.

The present invention discloses a nano antibody including CDR1, CDR2 and CDR3.

The amino acid sequence of CDR1 is SEQ ID NO: 9 FTFSSX₃A, where X₃ is selected from any one of F and Y.

The amino acid sequence of CDR2 is SEQ ID NO: 10 SGGGNT.

The amino acid sequence of CDR3 is SEQ ID NO: 11 TPGGRLWYYRYD.

In one embodiment according to the present invention, the amino acid sequence of the nano antibody is SEQ ID NO: 1, EVQLQASGGGFVQPGGSLRLSCAASGFTFSSX₁AMGWFRQAPGKEREX₂VSAISSGGGNTYYADSVKGRFTISRDNSKNTVYLQMNSLRAEDTATYYCVTPGGRLWYYRYDYRCQGTQVTVSS, where X₁ is selected from Y or F, and X₂ is selected from F or L.

In one embodiment according to the present invention, the amino acid sequence of the nano antibody is SEQ ID NO: 2, SEQ ID NO: 3 or SEQ ID NO: 4.

The present invention further discloses the use of the above-mentioned nano antibody in the preparation of a drug for treating a disease induced by Galectin-10 crystals.

In one embodiment according to the present invention, the disease induced by Galectin-10 crystals is selected from one or more of allergic disease, hyperimmune disease due to bacterial, fungal or viral infection, leukemia, tumor, and enteritis.

In one embodiment according to the present invention, the allergic disease is selected from one or more of asthma, allergic rhinosinusitis, allergic dermatitis, and eosinophilia.

The present invention further discloses a drug for treating an disease induced by Galectin-10 crystals, the drug containing the above-mentioned nano antibody and pharmaceutically acceptable excipients.

In one embodiment according to the present invention, the dosage form of the drug is selected from one or more of injection, spray, aerosol, powder spray or drop.

Another aspect of the present invention further relates to the use of the above-mentioned nano antibody in the treatment of a disease induced by Galectin-10 crystals, where the disease induced by Galectin-10 crystals is selected from one or more of allergic disease, hyperimmune disease due to bacterial, fungal or viral infection, leukemia, tumor, and enteritis; and further, the allergic disease is selected from one or more of asthma, allergic rhinosinusitis, allergic dermatitis, and eosinophilia.

The present invention further discloses the use of the above-mentioned nano antibody in the preparation of a test reagent for testing Charcot-Leyden crystals and/or Galectin-10 protein.

Further, the use includes the preparation of a diagnostic reagent for a disease with Charcot-Leyden crystals as test markers; and the disease with Charcot-Leyden crystals as test markers includes one or more of paragonimiasis westermani, paragonimiasis, amebic dysentery, eosinophilic gastroenteritis, or bronchial asthma.

The nano antibody of the present invention can also be used for in vitro test of Charcot-Leyden crystals and Galectin-10 protein, including scientific research use and diagnostic use. The Charcot-Leyden crystals and/or the Galectin-10 protein are also test markers for various diseases, and can be used for auxiliary clinical diagnosis of parasitic diseases such as paragonimiasis westermani, paragonimiasis and amoebic dysentery, and auxiliary clinical diagnosis of eosinophilic gastroenteritis, bronchial asthma, atopic dermatitis in children, etc.

The present invention has the following beneficial effects:

The nano antibody provided by the present invention can rapidly dissolve CLCs, and can significantly reduce pulmonary inflammation, changes in lung function and mucus production. The nano antibody can be used to treat allergic diseases such as asthma, allergic sinusitis, allergic dermatitis and eosinophilia affected by CLCs, hyperimmune diseases caused by bacterial, fungal or viral infections, and leukemia, tumor, enteritis, etc.

### BRIEF DESCRIPTION OF THE DRAWINGS

FIG. 1 is a plasmid profile of a Galectin-10 protein expression vector PK300.

FIG. 2 is a page gel diagram of Pk300 recombinant protein before and after EK digestion, where lane 1 is before EK digestion; lane 2 is after EK digestion; GST+Galectin-10 is 44.6 kDa, Galectin-10 is 16.8 kDa, and the loading volume is 5 µL.

FIG. 3 is an affinity chromatogram of purified Galectin-10 protein.

FIG. 4 is a diagram of SDS-PAGE electrophoresis results of purified Galectin-10 protein, where, M: protein molecular weight standard; 1: whole bacteria; 2: precipitate; 3: loading sample (whole bacteria broken supernatant); 4: flow through; 5: wash impurities with eluent; 6: 50% buffer B peak; 7: 50% buffer B; 8: 50% buffer B peak; 9-12: 100% buffer B.

FIG. 5 is a diagram of SDS-PAGE electrophoresis results of 172# nano antibody (SEQ ID NO: 4), where, M: protein molecular weight standard; 1: whole bacteria; 2: precipitate; 3: loading sample (whole bacteria broken supernatant); 4: flow through; 5-8: 30% buffer B; 9-11: 100% buffer B.

FIG. 6 is a diagram of SDS-PAGE electrophoresis results of 254# nano antibody (SEQ ID NO: 2), where, M: protein molecular weight standard; 1: whole bacteria; 2: precipitate; 3: loading sample (whole bacteria broken supernatant); 4: flow through; 5: wash with eluent; 6: 5% buffer B peak; 7: 5% buffer B; 8: 5% buffer B peak; 9: 100% buffer B; 10: 100% buffer B; 11: 100% buffer B.

FIG. 7 is a diagram of SDS-PAGE electrophoresis results of recombinant protein of 225# nano antibody (SEQ ID NO: 2), where, M: protein molecular weight standard; 1: precipitate; 2: loading sample (whole bacteria broken supernatant); 3: flow through; 4-11: 30% buffer B;12-14:100% buffer B.

FIG. 8 is a diagram of SDS-PAGE electrophoresis results of 225# nano antibody (SEQ ID NO: 4), where, M: protein molecular weight standard; 1: whole bacteria; 2: precipitate; 3: loading sample (whole bacteria broken supernatant); 4: flow through; 5-8: 30% buffer B; 9-11: 100% buffer B.

FIG. 9 shows a microscope view of Galectin-10 crystals.

FIG. 10 is a diagram of the process of dissolving crystals by 172# nano antibody (SEQ ID NO: 2).

FIG. 11 is a diagram of the process of dissolving crystals by 254# nano antibody (SEQ ID NO: 3).

FIG. 12 is a diagram of the process of dissolving crystals by 225# nano antibody (SEQ ID NO: 4).

FIG. 13 is a diagram of the buffer control in the process of dissolving crystals.

FIG. 14 is a plasmid profile of a protein expression vector expressing 172# nano antibody.

FIG. 15 is a plasmid profile of a protein expression vector expressing 254# nano antibody.

FIG. 16 is a plasmid profile of a protein expression vector expressing 225# nano antibody.

FIG. 17 is a plasmid profile of Galectin-10 protein expression vector PK277.

FIG. 18 is a diagram of SDS-PAGE electrophoresis results of purified Galectin-10 protein with His tag, where, M: protein molecular weight standard; 1: whole bacteria; 2: precipitate; 3: loading sample (whole bacteria broken supernatant); 4: flow through; 5: wash with eluent; 6-9: elution peaks at different concentration gradients.

### DETAILED DESCRIPTION

The following embodiments are used to illustrate the present application, but are not intended to limit the scope of the present application.

Specific embodiments of the present application will be described in more detail below. These embodiments are provided so that the present application can be understood more thoroughly, and can fully convey the scope of the present application to those skilled in the art.

As referred to throughout the specification and claims, the term “include” or “comprise” is an open-ended term and should be interpreted as “include but not limited to”. Subsequent descriptions in the specification are preferred embodiments for implementing the present application, however, the descriptions are for the purpose of general principles of the specification and are not intended to limit the scope of the present application. The protection scope of the present application should be determined by the appended claims.

### Embodiment 1 Expression and Purification of Galectin-10 Protein

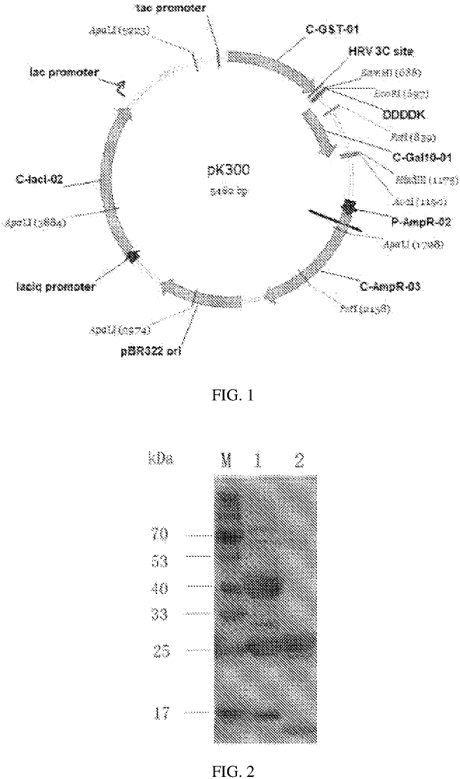

The amino acid sequence of Galectin-10 protein is shown as SEQ ID NO: 5. A PK300 plasmid profile of a Galectin-10 protein expression vector is shown in FIG. 1, and FIG. 2 shows an electrophoresis diagram of Pk300 recombinant protein before and after EK digestion, where GST+Galectin-10 is 44.6 kDa, Galectin-10 is 16.8 kDa, and the loading volume is 5 µL. After an engineering strain LB liquid medium containing PK300 plasmids is cultured overnight at 37° C. while shaking, the engineering strain is inoculated with an inoculum of 1:100 into a shake flask containing 1 L of LB medium and cultured at 37° C. for 2 h, isopropyl-β-D-thiogalactoside (IPTG) with a final concentration of 1 mmol/ L is added for induction, and cooling to 30° C. is performed for overnight culture. Bacteria are collected by centrifugation at 8000 r/min for 10 min, resuspended with a bacteria breaking solution (20 mM PB, 0.3 M NaCl, pH 7.9) and then sonicated, the broken solution is centrifuged at 12000 r/min for 30 min, and the supernatant is taken for next purification.

Since the front-end sequence of the recombinant protein contains GST fusion protein, the recombinant protein can be purified by an affinity chromatography column. The buffer used for affinity chromatography is 20 mM PB, pH=7.4, and the buffer used for elution is 1×PBS (10 mM PB, 136 mM NaCl), 100 mM GSH, pH=7.4. The eluted fractions are collected, and the expression of the target protein is tested by sodium dodecyl sulfate-polyacrylamide gel electrophoresis (SDS-PAGE).

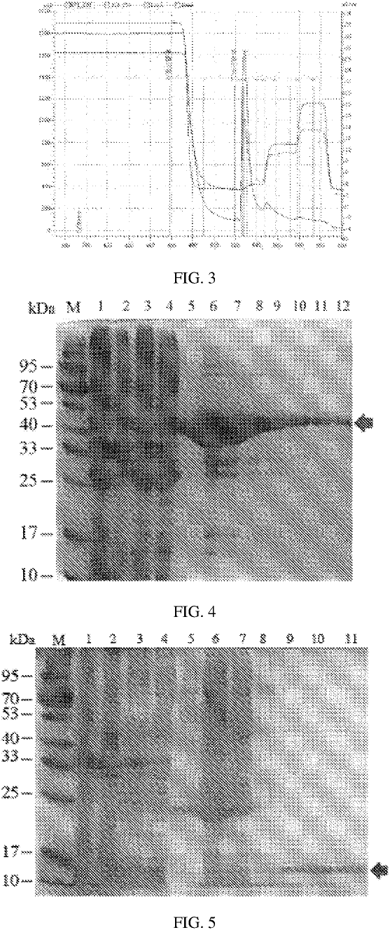

The supernatant is purified by a GST affinity chromatography column. The chromatogram is shown in FIG. 3. The elution peak appears in 50% buffer B alone, indicating that the protein binds effectively to GST. The flow-through peaks and elution peaks are collected separately and subjected to SDS-PAGE electrophoresis. The results are shown in FIG. 4.

### Embodiment 2 Expression of Recombinant Protein of Nano Antibody

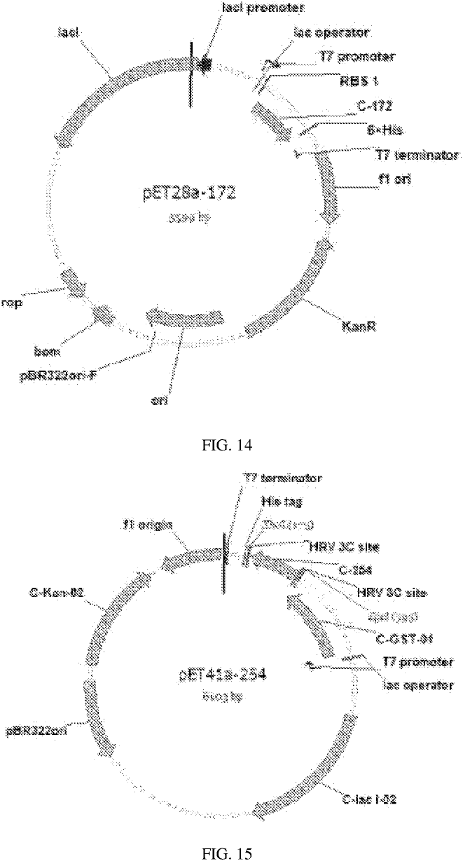

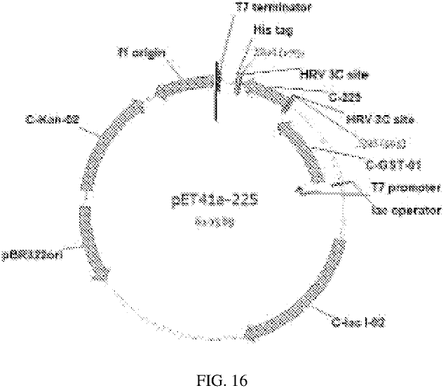

The present invention is based on coding genes (SEQ ID NO: 6, SEQ ID NO: 7, SEQ ID NO: 8) of nano antibodies of which the amino acid sequences are respectively SEQ ID NO: 1, SEQ ID NO: 2 and SEQ ID NO: 3 to construct expression vectors. Specifically, designing primers to amplify coding genes and then enzymatically digesting, a gene coding sequence is constructed on a protein expression vector by means of enzymatic digestion and T4 link. As shown in FIG. 14, FIG. 15 and FIG. 16, the gene sequence of the 172# nano antibody is linked to the expression vector pET28a, and the 225 and 254 sequences are respectively constructed on the protein expression vector Pet41b. Then the bacteria are shaken to induce protein expression, and the recombinant protein is cultured and expressed by engineering strains containing expression vectors respectively. Specifically, after engineering strains are cultured in an LB liquid medium overnight at 37° C. while shaking, the strains are inoculated with an inoculum of 1:100 into a shake flask containing 1 L of LB medium and cultured at 37° C. for 2 h, isopropyl-β-D-thiogalactoside (IPTG) with a final concentration of 1 mmol/ L is added for induction, and cooling to 16° C. is performed for overnight culture. Bacteria are collected by centrifugation at 8000 r/min for 10 min, resuspended with a bacteria breaking solution (20 mM PB, 150 mM NaCl, pH 8.0) and then sonicated, the broken solution is centrifuged at 12000 r/min for 30 min, and the supernatant is taken for next purification.

Since the front-end sequence of the recombinant protein contains GST fusion protein, the recombinant protein can be purified by a GST affinity chromatography column. The buffer used for affinity chromatography is 20 mM PB, 150 mM NaCl, pH=7.5, and the buffer used for elution is 20 mM PB, 150 mM NaCl, 100 mM GSH, pH=7.5. The eluted fractions are collected, and the expression of the target protein is tested by sodium dodecyl sulfate-polyacrylamide gel electrophoresis (SDS-PAGE).

The results are as follows:

The supernatant of the recombinant protein of the 172# nano antibody (SEQ ID NO: 2) is purified by a His affinity chromatography column, the size of the target protein eluted by 100% buffer B is consistent with that of the recombinant protein (14.495 kDa), the recombinant protein (lane 10) has a purity of 80% and a concentration of 0.5 mg/mL, and the results of SDS-PAGE electrophoresis are shown in FIG. 5.

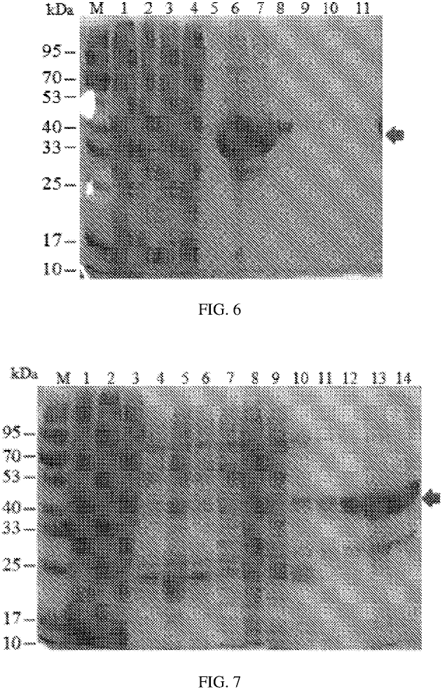

The supernatant of the recombinant protein of the 254# nano antibody (SEQ ID NO: 3) is purified by a GST affinity chromatography column, the size of the target protein of 50% buffer B peak is consistent with that of the recombinant protein (42.54 kDa), and the recombinant protein (sample in lane 6) has a purity of 80% and a concentration of 3 mg/mL. The results of SDS-PAGE electrophoresis are shown in FIG. 6.

The recombinant protein of the 225# nano antibody (SEQ ID NO: 4) is purified by a His affinity chromatography column. The buffer used for affinity chromatography is 20 mM PB, 150 mM NaCl, pH=7.5, and the buffer used for elution is 20 mM PB, 150 mM NaCl, 500 mM imidazole, pH=7.5. The eluted fractions are collected, and the expression of the target protein is tested by sodium dodecyl sulfate-polyacrylamide gel electrophoresis (SDS-PAGE). The results of SDS-PAGE electrophoresis are shown in FIG. 7.

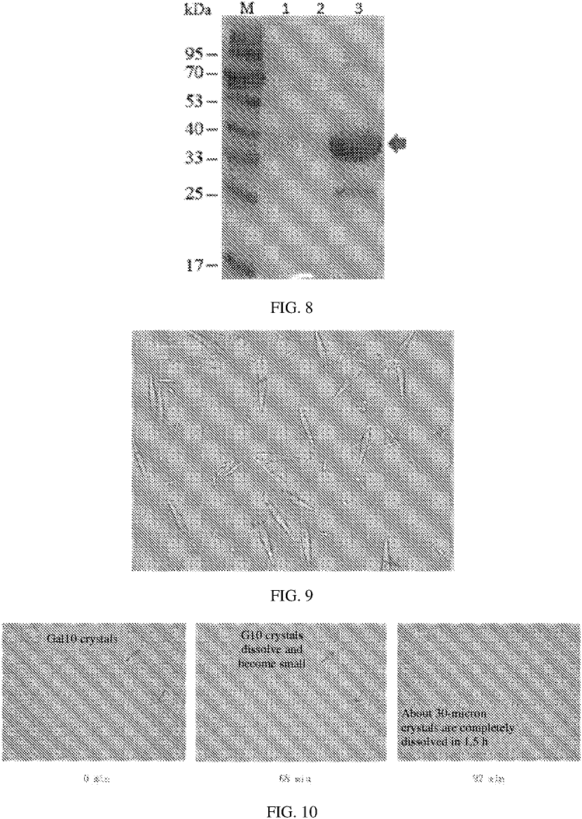

The concentration of the recombinant protein is low after the elution of 100% buffer B by His affinity chromatography, and the front-end sequence of the recombinant protein contains GST fusion protein, so eluent 13 and eluent 14 can be purified again by GST affinity chromatography. The results of SDS-PAGE electrophoresis are shown in FIG. 8.

### Embodiment 3 Test on Galectin-10 Protein Crystals

1. The PK300 vector expressing the Galectin-10 recombinant protein (Galectin-10 protein with GST tag 5 mg/mL) (the pk300 vector profile is shown in FIG. 1) is digested with an EK enzyme (purchased from Sino Biological Inc., article number S019100750) at 20° C. for 16 hours according to the following proportions. The electrophoresis diagram before and after digestion is shown in FIG. 2. The size of the GST tag + Gal protein before digestion is 44.6 kDa, but the protein is partially degraded. After digestion, the size of the Galectin-10 protein is 16.8 kDa. The band below indicated by the red arrow is Galectin-10 protein.

| | |
|---|---|
| Buffer | 60 µL |
| PK300 recombinant protein | 516 µL |
| EK enzyme | 24 µL |
| Total volume | 600 µL |

2. Then 50% PEG3350 and 5 × PBS are added according to the following volumes. Precipitation is performed for 16 hours at 20° C.

| | |
|---|---|
| Digestion product | 560 µL |
| 5 × PBS | 70 µL |
| 50% PEG3350 | 70 µL |
| Total volume | 700 µL |

3. Centrifugation is performed at 6000 rpm for 5 min, the supernatant is discarded, 500 µL of 1 × PBS is added, followed by uniform mixing.

Step 3 is repeated twice, then 100 µL of 1 × PBS is added, followed by uniform mixing to obtain Galectin-10 crystals for dissolution test. The obtained crystals are observed under a microscope as shown in FIG. 9, where there are a large number of typical CLCs.

### Embodiment 4 Dissolution Scheme of Crystals

3 µL of Galectin-10 crystals are pipetted into a 1.5 mL centrifuge tube, then 30 µL of Galectin-10 antibody to be tested is added, shaken for uniform mixing, and 10 µL are immediately pipetted and placed under a microscope for microscopic examination, and pictures are taken at regular intervals until all the crystals in the field of view are completely dissolved, and the time required for dissolution is recorded. During this period, the microscope cannot be shaken and adjusted to prevent the field of view from changing.

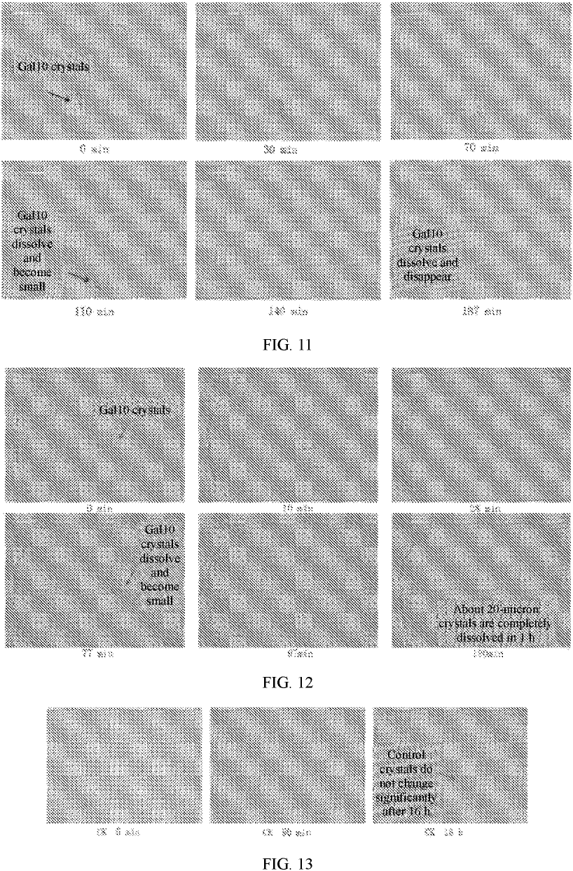

The dissolution process is shown in FIGS. 10-13. Wherein FIG. 10 is a process diagram of dissolving crystals by 172# nano antibody (SEQ ID NO: 2); FIG. 11 is a process diagram of dissolving crystals by 254# nano antibody (SEQ ID NO: 3); FIG. 12 is a process diagram of dissolving crystals by 225# nano antibody (SEQ ID NO: 4); and FIG. 13 is a buffer control diagram in the process of dissolving crystals. It can be seen that the three nano antibodies provided by the present invention can dissolve Gal10 crystals quickly and effectively.

### Embodiment 5 Test on Galectin-10 Protein Content

### I. Extraction and Purification of Galectin-10 Protein.

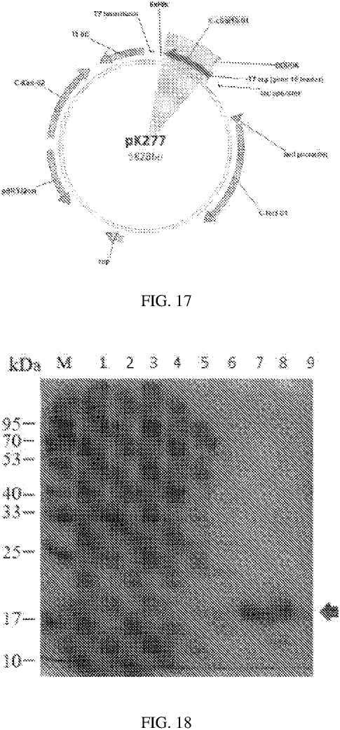

The amino acid sequence of Galectin-10 protein is shown as SEQ ID NO: 5. The plasmid profile of Galectin-10 protein expression vector PK277 is shown in FIG. 17. After an engineering strain LB liquid medium containing PK277 plasmids is cultured overnight at 37° C. while shaking, the strain is inoculated with an inoculum of 1:100 into a shake flask containing 1 L of LB medium and cultured at 37° C. for 2 h, isopropyl-β-D-thiogalactoside (IPTG) with a final concentration of 1 mmol/ L is added for induction, and cooling to 30° C. is performed for overnight culture. Bacteria are collected by centrifugation at 8000 r/min for 10 min, resuspended with a bacteria breaking solution (20 mM PB, 0.3 M NaCl, pH 7.9) and then sonicated, the broken solution is centrifuged at 12000 r/min for 30 min, and the supernatant is taken for next purification.

The results are as follows:

The supernatant of the recombinant protein of the Galectin-10 with His tag (SEQ ID NO: 2) is purified by a His affinity chromatography column, the size of the target protein eluted is consistent with that of the recombinant protein (21.3 kDa), the recombinant protein (lane 10) has a purity of 80% and a concentration of 0.3 mg/mL, and the results of SDS-PAGE electrophoresis are shown in FIG. 18. FIG. 18 shows an electrophoresis diagram of Pk277 recombinant protein, where His tag+Galectin-10 is 21.3 kDa, and the loading volume is 5 µL.

### II. Test on Galectin-10 Protein Content Using 172# Nano Antibody

### 1. Test Equipment

Incubator, plate washer, microplate reader.

### 2. Test Reagents

Galectin-10 protein, Galectin-10 monoclonal antibody (including His tag antibody and GST tag antibody), HRP-tagged anti-His tag antibody, anti-GST tag mouse monoclonal antibody, HRP-tagged goat anti-mouse antibody, TMB single-component developing solution.

### 3. Scope of Application of the Test Scheme

All Galectin-10 ELISA assays.

### 4. Test Method

(1) Phosphate buffered saline (PBS): weigh 8.0 g of NaCl, 0.2 g of KH₂PO₄, and 2.96 g of Na₂HPO₄•12H₂O, add 1000 ml of distilled water with a measuring cylinder, pH 7.5.

(2) Sample diluent (PBST): add 0.1 ml of Tween-20 to 100 ml of PBS.

(3) Washing solution (PBST): add 1 ml of Tween-20 to 1000 ml of PBS.

(4) Stop solution: 1 mol/L H₂SO₄. Note that sulfuric acid is added to water with constant stirring. This reaction releases a lot of heat.

(5) Coating buffer: weigh 1.5 g of Na₂CO₃, 2.93 g of NaHCO₃, and 0.2 g of NaN₃ (optional), add 1000 ml of distilled water with a measuring cylinder, pH 9.6.

(6) Blocking solution: dissolve 3 g of bovine serum albumin in 100 ml of coating buffer.

### 5. Sample Determination (His Tag Antibody)

1) Coating: dilute 3 mg/mL Galectin-10 protein with the coating buffer to 1:500, 1:2000, and 1:10000, 100 ul per well, 3 replicate wells for each concentration, 4° C., coat in a wet box overnight. Details are shown in Table 1.

TABLE 1

Galectin-10 protein coating concentration table

| | | | |
|---|---|---|---|
| | 1 | 2 | 3 |
| A | Uncoated | Uncoated | Uncoated |
| B | 1: 500 | 1: 500 | 1: 500 |
| C | 1: 2000 | 1: 2000 | 1: 2000 |
| D | 1: 10000 | 1: 10000 | 1: 10000 |

2) Wash the plate: shake off the sample, add 300 ul of washing solution with a multichannel pipette and then pour out, wash the plate 4 times, and drop the plate on newspaper twice.

3) Block: add 120 ul of blocking solution to each well, place in a wet box at 37° C. for 2 h, and then shake off the blocking solution.

4) Add His tag 172# antibody for 1:200 dilution, 100 ul per well, incubate at 37° C. for 1 h in a wet box.

5) Wash the plate: same as 2).

6) Add HRP-tagged anti-His tag antibody for 1:1000 dilution, 100 ul per well, incubate at 37° C. for 1 h in a wet box.

7) Wash the plate: same as 2).

8) Add the TMB single-component developing solution, 100 ul per well, dark for 5 min, and add an equal volume of stop solution.

9) Perform colorimetric reading at 450 nm wavelength with the microplate reader.

TABLE 2

172# nano antibody test results

| Concentration of coated Galectin-10 | Repeat 1 | Repeat 2 | Repeat 3 | Average |
|---|---|---|---|---|
| 0 | 0.428 | 0.456 | 0.452 | 0.445±0.09 |
| 6000 ng | 1.771 | 1.848 | 1.877 | 1.832±0.032 |
| 1500 ng | 1.767 | 1.687 | 1.72 | 1.725±0.023 |
| 300 ng | 1.126 | 1.196 | 1.496 | 1.273±0.113 |

From the ELISA test results, the measured OD values of the Galectin-10 protein at 6000 ng/mL, 1500 ng/mL and 300 ng/mL are significantly different from those of uncoated Galectin-10 protein test wells. It can be determined that the minimum test concentration of the Galectin-10 protein tested with the 172# nano antibody is 300 ng/mL. It is proved that the 172# nano antibody can be used for the test of Galectin-10 protein (for scientific research use and clinical test use, etc.).

### III. Test on Galectin-10 Protein Content Using 254# and 225# Nano Antibodies.

### 1. Test Equipment

Incubator, plate washer, microplate reader.

### 2. Test Reagents

Galectin-10 protein with His tag (expressed and purified with pk277, see Annex 1 for expression and purification methods), Galectin-10 nano antibodies 254# and 225# (antibodies with GST tags), anti-GST tag mouse monoclonal antibody, HRP-tagged goat anti-mouse antibody, TMB single-component developing solution.

### 3. Scope of Application of the Test Scheme

All Galectin-10 ELISA assays.

### Test Method

(1) Phosphate buffered saline (PBS): weigh 8.0 g of NaCl, 0.2 g of KH₂PO₄, and 2.96 g of Na₂HPO₄•12H₂O, add 1000 ml of distilled water with a measuring cylinder, pH 7.5.

(2) Sample diluent (PBST): add 0.1 ml of Tween-20 to 100 ml of PBS.

(3) Washing solution (PBST): add 1 ml of Tween-20 to 1000 ml of PBS.

(4) Stop solution: 1 mol/L H₂SO₄. Note that sulfuric acid is added to water with constant stirring. This reaction releases a lot of heat.

(5) Coating buffer: weigh 1.5 g of Na₂CO₃, 2.93 g of NaHCO₃, and 0.2 g of NaN₃ (optional), add 1000 ml of distilled water with a measuring cylinder, pH 9.6.

(6) Blocking solution: dissolve 3 g of bovine serum albumin in 100 ml of coating buffer.

### 4. Sample Determination (GST Tag Antibody)

1) Coating: dilute 0.25 mg/mL Galectin-10 protein with the coating buffer to 0 well, 1:100, 1:500, and 1:2000, 100 ul per well, 3 replicate wells for each concentration, 4° C., coat in a wet box overnight, as shown in Table 3.

TABLE 3

Galectin-10 protein coating table

| | | | | | | |
|---|---|---|---|---|---|---|
| | 1 | 2 | 3 | 4 | 5 | 6 |
| A | Uncoated | Uncoated | Uncoated | Uncoated | Uncoated | Uncoated |
| B | 1: 100 | 1: 100 | 1: 100 | 1: 100 | 1: 100 | 1: 100 |
| C | 1: 500 | 1: 500 | 1: 500 | 1: 500 | 1: 500 | 1: 500 |
| D | 1: 2000 | 1: 2000 | 1: 2000 | 1: 2000 | 1: 2000 | 1: 2000 |

2) Wash the plate: shake off the sample, add 300 ul of washing solution with a multichannel pipette and then pour out, wash the plate 4 times, and drop the plate on newspaper twice.

3) Block: add 120 ul of blocking solution to each well, place in a wet box at 37° C. for 2 h, and then shake off the blocking solution.

4) Add GST tag antibodies 254# and 225#, dilute 1:200, 100 ul per well, add 254# to 1, 2, and 3 columns, and 225# to 4, 5, and 6 columns, and incubate at 37° C. for 1 h in a wet box.

5) Wash the plate: same as 2).

6) Add anti-GST tag mouse monoclonal antibody, dilute 1:500, 100 ul per well, and incubate at 37° C. for 1 h in a wet box.

7) Wash the plate: same as 2).

8) Add HRP-tagged goat anti-mouse antibody, dilute 1:500, 100 ul per well, and incubate at 37° C. for 1 h in a wet box.

9) Wash the plate: same as 2).

10) Add the TMB single-component developing solution, 100 ul per well, dark for 20 min, and add an equal volume of stop solution.

11) Perform colorimetric reading at 450 nm wavelength with the microplate reader. The results are shown in Table 4 and Table 5.

TABLE 4

254# antibody test results

| Concentration of Galectin-10 (ng/mL) | Repeat 1 | Repeat 2 | Repeat 3 | Average |
|---|---|---|---|---|
| 0 | 0.328 | 0.308 | 0.312 | 0.316±0.006 |
| 2500 | 1.317 | 1.198 | 1.34 | 1.285±0.044 |
| 500 | 0.615 | 0.551 | 0.577 | 0.581±0.019 |
| 125 | 0.397 | 0.353 | 0.37 | 0.373±0.013 |

TABLE 5

225# antibody test results

| Concentration of Galectin-10 (ng/mL) | Repeat 1 | Repeat 2 | Repeat 3 | Average |
|---|---|---|---|---|
| 0 | 0.119 | 0.134 | 0.128 | 0.127±0.004 |
| 2500 | 0.74 | 0.7 | 0.681 | 0.707±0.017 |
| 500 | 0.257 | 0.26 | 0.279 | 0.265±0.007 |
| 125 | 0.155 | 0.153 | 0.173 | 0.160±0.006 |

From the ELISA test results shown in Table 4 and Table 5, when the Galectin-10 protein is at 2500 ng/mL, 500 ng/mL and 125 ng/mL, the measured OD values of the 254# and 225# nano antibodies are significantly different from those of uncoated Galectin-10 protein test wells. It can be determined that the minimum test concentration of the two antibodies is 125 ng/mL. It is proved that the 254# and 225# nano antibodies can be used for the test of Galectin-10 protein. This test method is only an example, but is not limited.

Although the present application is described in detail above with general descriptions and specific embodiments, some modifications or improvements can be made on the basis of the present application, which is obvious to those skilled in the art. Therefore, all these modifications or improvements made without departing from the spirit of the present application fall into the scope of the present application.

### SEQUENCE TABLE

Seq Id No: 1

EVQLQASGGGFVQPGGSLRLSCAASGFTFSSX₁AMGWFRQAPGKEREX₂V
SAISSGGGNTYYADSVKGRFTISRDNSKNTVYLQMNSLRAEDTATYYCVT
PGGRLWYYRYDYRCQGTQVTVSS;

Herein, X₁ is selected from Y or F, and X₂ is selected from F or L

172# Nano Antibody (SEQ ID NO: 2)

EVQLQASGGGFVQPGGSLRLSCAASGFTFSSYAMGWFRQAPGKEREFVSA
ISSGGGNTYYADSVKGRFTISRDNSKNTVYLQMNSLRAEDTATYYCVTPG
GRLWYYRYDYRCQGTQVTVSS

254# Nano Antibody (SEQ ID NO: 3)

EVQLQASGGGFVQPGGSLRLSCAASGFTFSSFAMGWFRQAPGKEREFVSA
ISSGGGNTYYADSVKGRFTISRDNSKNTVVLQMNSLRAEDTATYYCVTPG
GRLWYYRYDYRCQGTQVTVSS

225# Nano Antibody (SEQ ID NO: 4)

EVQLQASGGGFVQPGGSLRLSCAASGFTFSSYAMGWFRQAPGKERELVSA
ISSGGGNTYYADSVKGRFTISRDNSKNTVYLQMNSLRAEDTATYYCVTPG
GRLWYYRYDYRCQGTQVTVSS

Galectin-10 (SEQ ID NO: 5)

MSLLPVPYTEAASLSTGSTVTIKGRPLACFLNEPYLQVDFHTEMKEESDI
VFHFQVCFGRRVVMNSREYGAWKQQVESKNMPFQDGQEFELSISVLPDKY
QVMVNGQSSYTFDHRIKPEAVKMVQVWRDISLTKFNVSYLKR

Coding Gene of 172# Nano Antibody SEQ ID NO: 6

GAGGTGCAGC TGCAAGCGAG CGGTGGCGGT TTCGTTCAAC CGGGCGGTAG
CCTGCGTCTG AGCTGCGCGG CGAGCGGTTT CACCTTTAGC AGCTACGCGA
TGGGTTGGTT CCGTCAAGCG CCGGGCAAGG AGCGTGAATT TGTTAGCGCG
ATCAGCAGCG GCGGTGGCAA CACCTACTAT GCGGACAGCG TGAAGGGTCG
TTTTACCATT AGCCGTGATA ACAGCAAAAA CACCGTTTAC CTGCAAATGA
ACAGCCTGCG TGCGGAAGAC ACCGCGACCT ACTATTGCGT GACCCCGGGT
GGCCGTCTGT GGTACTATCG TTACGATTAT CGGTGTCAGG GCACCCAAGT
GACCGTTAGC AGC

Coding Gene of 254# Nano Antibody SEQ ID NO: 7

GAGGTGCAGC TGCAAGCGAG CGGTGGCGGT TTCGTTCAAC CGGGCGGTAG
CCTGCGTCTG AGCTGCGCGG CGAGCGGTTT CACCTTTAGC AGCTTCGCGA
TGGGTTGGTT CCGTCAAGCT CCGGGCAAGG AGCGTGAATT TGTTAGCGCG
ATCAGCAGCG GCGGTGGCAA CACCTACTAT GCGGACAGCG TGAAGGGTCG
TTTTACCATT AGCCGTGATA ACAGCAAAAA CACCGTTTAC CTGCAAATGA
ACAGCCTGCG TGCGGAAGAC ACCGCGACCT ACTATTGCGT GACCCCGGGT
GGCCGTCTGT GGTACTATCG TTACGATTAT CGGTGTCAGG GCACCCAAGT
GACCGTTAGC AGC

Coding Gene of 225# Nano Antibody SEQ ID NO: 8

GAGGTGCAGC TGCAAGCGAG CGGTGGCGGT TTCGTTCAAC CGGGCGGTAG
CCTGCGTCTG AGCTGCGCGG CGAGCGGTTT CACCTTTAGC AGCTACGCGA
TGGGTTGGTT CCGTCAAGCG CCGGGCAAGG AGCGTGAACT TGTTAGCGCG
ATCAGCAGCG GCGGTGGCAA CACCTACTAT GCGGACAGCG TGAAGGGTCG
TTTTACCATT AGCCGTGATA ACAGCAAAAA CACCGTTTAC CTGCAAATGA
ACAGCCTGCG TGCGGAAGAC ACCGCGACCT ACTATTGCGT GACCCCGGGT
GGCCGTCTGT GGTACTATCG TTACGATTAT CGGTGTCAGG GCACCCAAGT
GACCGTTAGC AGC

Amino Acid Sequence of CDR1 SEQ ID NO: 9

FTFSSX₃A

Herein, X₃ is F or Y

Amino Acid Sequence of CDR2 SEQ ID NO: 10

SGGGNT

Amino Acid Sequence of CDR3 SEQ ID NO: 11

TPGGRLWYYRYD

1. A nano antibody, wherein the nano antibody comprises CDR1, CDR2 and CDR3; the amino acid sequence of CDR1 is SEQ ID NO: 9 FTFSSX₃A, where X₃ is selected from any one of F and Y;
the amino acid sequence of CDR2 is SEQ ID NO: 10 SGGGNT; and
the amino acid sequence of CDR3 is SEQ ID NO: 11 TPGGRLWYYRYD.

2. The nano antibody according to claim 1, wherein the amino acid sequence of the nano antibody is SEQ ID NO: 1,
EVQLQASGGGFVQPGGSLRLSCAASGFTFSSX₁AMGWFRQAPGKEREX₂VSAISSGGGNTYYADSVKGRFTISRDNSKNTVYLQMNSLRAEDTATYYCVTPGGRLWYYRYDYRCQGTQVTVSS, where X₁ is selected from Y or F, and X₂ is selected from F or L.

3. The nano antibody according to claim 2, wherein the amino acid sequence of the nano antibody is SEQ ID NO: 2, SEQ ID NO: 3 or SEQ ID NO: 4.

4. The nano antibody according to claim 3, wherein the coding gene of the nano antibody is SEQ ID NO: 6, SEQ ID NO: 7 or SEQ ID NO: 8.

5. The use of the nano antibody according to claim 1 in the preparation of a drug for treating a disease induced by Charcot-Leyden crystals (CLCs).

6. The use of the nano antibody according to claim 5, wherein the disease induced by CLCs is selected from one or more of allergic disease, hyperimmune disease due to bacterial, fungal or viral infection, leukemia, tumor, and enteritis.

7. The use of the nano antibody according to claim 6, wherein the allergic disease is selected from one or more of asthma, allergic rhinosinusitis, allergic dermatitis, and eosinophilia.

8. A drug for treating an disease induced by CLCs , wherein the drug contains the nano antibody according to claim 1 and pharmaceutically acceptable excipients.

9. The use of the nano antibody according to claim 1 in the preparation of a reagent for testing Charcot-Leyden crystals (CLCs) and/or Galectin-10 protein.

10. The use of the nano antibody according to claim 9, wherein the use comprises the preparation of a diagnostic reagent for a disease with Charcot-Leyden crystals as test markers; and the disease with Charcot-Leyden crystals as test markers comprises one or more of paragonimiasis westermani, paragonimiasis, amebic dysentery, eosinophilic gastroenteritis, atopic dermatitis in children, or bronchial asthma.


SEQUENCE LISTING (11 sequences)

SEQ ID NO: 1 - protein, 121 aa, Artificial Sequence
  This sequence is synthesized in lab.
  VARIANT (32)..(32): F or Y
  VARIANT (47)..(47): F or L
EVQLQASGGGFVQPGGSLRLSCAASGFTFSSXAMGWFRQAPGKEREXVSAISSGGGNTYYADSVKGRFTISRDNSKNTVYLQMNSLRAEDTATYYCVTPGGRLWYYRYDYRCQGTQVTVSS

SEQ ID NO: 2 - protein, 121 aa, Artificial Sequence
  This sequence is synthesized in lab.
EVQLQASGGGFVQPGGSLRLSCAASGFTFSSYAMGWFRQAPGKEREFVSAISSGGGNTYYADSVKGRFTISRDNSKNTVYLQMNSLRAEDTATYYCVTPGGRLWYYRYDYRCQGTQVTVSS

SEQ ID NO: 3 - protein, 121 aa, Artificial Sequence
  This sequence is synthesized in lab.
EVQLQASGGGFVQPGGSLRLSCAASGFTFSSYAMGWFRQAPGKERELVSAISSGGGNTYYADSVKGRFTISRDNSKNTVYLQMNSLRAEDTATYYCVTPGGRLWYYRYDYRCQGTQVTVSS

SEQ ID NO: 4 - protein, 121 aa, Artificial Sequence; identical to SEQ ID NO: 3
  This sequence is synthesized in lab.

SEQ ID NO: 5 - protein, 142 aa, Artificial Sequence
  This sequence is synthesized in lab.
MSLLPVPYTEAASLSTGSTVTIKGRPLACFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKFNVSYLKR

SEQ ID NO: 6 - DNA, 363 bp, Artificial Sequence
  This sequence is synthesized in lab.
gaggtgcagctgcaagcgagcggtggcggtttcgttcaaccgggcggtagcctgcgtctgagctgcgcggcgagcggtttcacctttagcagctacgcgatgggttggttccgtcaagcgccgggcaaggagcgtgaatttgttagcgcgatcagcagcggcggtggcaacacctactatgcggacagcgtgaagggtcgttttaccattagccgtgataacagcaaaaacaccgtttacctgcaaatgaacagcctgcgtgcggaagacaccgcgacctactattgcgtgaccccgggtggccgtctgtggtactatcgttacgattatcggtgtcagggcacccaagtgaccgttagcagc

SEQ ID NO: 7 - DNA, 363 bp, Artificial Sequence
  This sequence is synthesized in lab.
gaggtgcagctgcaagcgagcggtggcggtttcgttcaaccgggcggtagcctgcgtctgagctgcgcggcgagcggtttcacctttagcagcttcgcgatgggttggttccgtcaagctccgggcaaggagcgtgaatttgttagcgcgatcagcagcggcggtggcaacacctactatgcggacagcgtgaagggtcgttttaccattagccgtgataacagcaaaaacaccgtttacctgcaaatgaacagcctgcgtgcggaagacaccgcgacctactattgcgtgaccccgggtggccgtctgtggtactatcgttacgattatcggtgtcagggcacccaagtgaccgttagcagc

SEQ ID NO: 8 - DNA, 363 bp, Artificial Sequence
  This sequence is synthesized in lab.
gaggtgcagctgcaagcgagcggtggcggtttcgttcaaccgggcggtagcctgcgtctgagctgcgcggcgagcggtttcacctttagcagctacgcgatgggttggttccgtcaagcgccgggcaaggagcgtgaacttgttagcgcgatcagcagcggcggtggcaacacctactatgcggacagcgtgaagggtcgttttaccattagccgtgataacagcaaaaacaccgtttacctgcaaatgaacagcctgcgtgcggaagacaccgcgacctactattgcgtgaccccgggtggccgtctgtggtactatcgttacgattatcggtgtcagggcacccaagtgaccgttagcagc

SEQ ID NO: 9 - protein, 7 aa, Artificial Sequence
  This sequence is synthesized in lab.
  VARIANT (6)..(6): F or Y
FTFSSXA

SEQ ID NO: 10 - protein, 6 aa, Artificial Sequence
  This sequence is synthesized in lab.
SGGGNT

SEQ ID NO: 11 - protein, 12 aa, Artificial Sequence
  This sequence is synthesized in lab.
TPGGRLWYYRYD